(19) United States
(12) Patent Application Publication
(10) Pub. No.: US 2015/0080819 A1
(43) Pub. Date: Mar. 19, 2015

(54) DIAPER STRUCTURE CAPABLE OF WIRELESSLY DETECTING A SOAKING WET CONDITION THEREOF AND DIAPER DETECTION SYSTEM OF THE SAME

(71) Applicant: SINGLI TECHNOLOGY INC., New Taipei City (TW)

(72) Inventors: Sharon Lin Charna, Irvine, CA (US); Lee-Tsan LU, Taipei City (TW)

(73) Assignee: SINGLI TECHNOLOGY INC., New Taipei City (TW)

(21) Appl. No.: 14/098,851

(22) Filed: Dec. 6, 2013

(30) Foreign Application Priority Data
Sep. 14, 2013 (TW) 102133347

(51) Int. Cl. A61F 13/42

(52) U.S. Cl. CPC A61F 13/42; A61F 2013/424
USPC 604/361

(57) ABSTRACT

The present invention is to provide a diaper structure, which includes a diaper main body formed by a fabric layer, an anti-leak layer and an absorbent material layer sequentially from the inner side to outer side thereof, and a communication chip and a metal antenna attached to the outer surface of the anti-leak layer and connected with each other to form an RFID tag. Thus, when the absorbent material layer absorbs the urine passing through the fabric layer and the inner surface of the anti-leak layer is in contact with the urine on the surface of the absorbent material layer, the change in the relative permittivity of the anti-leak layer causes a change in the impedance value of the metal antenna and consequently a change in the wavelength of the electromagnetic wave signal transmitted by the RFID tag, thereby enabling rapid detection of a soaking wet condition of the diaper.

### FIELD OF THE INVENTION

The present invention relates to a diaper, more particularly to a diaper structure capable of wirelessly detecting a soaking wet condition thereof without significant modification of a conventional diaper structure.

### BACKGROUND OF THE INVENTION

With the advent of an aging society, many medical groups as well as the academic field and the related industries have devoted themselves to the research and development of geriatric care products in order to address the health care issues of the elderly, the sick, and other care receivers.

Elderly people are less agile and less responsive than when they were young, so assistance is often needed in their daily chores. For example, a senior citizen with urinary incontinence may wear diapers to avoid wetting their pants or bed sheets, and a diaper must be changed immediately when soaking wet with urine. However, changing a diaper is no easy matter. A diaper wearer must take off their pants before changing and put the pants back on after the diaper is changed. During the process, it is also necessary for the diaper wearer to shift the center of gravity of their body frequently in order to pull the legs of the pants out of or over their legs. In the foregoing process, a senior person may need to hold on to an object (e.g., a table or a wall) to keep balance, or they are very likely to sway and fall to the ground, causing injury to themselves. To reduce the chances of such accidents, it is highly desirable that an elderly person is assisted by a family member or caregiver when changing diapers.

In a nursing home, it is now common practice for a nurse to be in charge of the hygiene and daily lives of several elderly residents. Therefore, it is impossible for a nursing home resident to be always accompanied by the assigned nurse; the latter performs nursing actions (e.g., changing diapers) only when needed. If a senior resident is verbally challenged, unconscious, or mentally dull due to a disease (e.g., a stroke), they may have problem informing the nurse immediately when in need of a diaper change. Once a delayed diaper change becomes the norm, not only will the senior person in question feel very uncomfortable, but also other diseases such as diaper rash may occur. To solve this problem, diaper products capable of wetness detection have been used in nursing homes. These diaper products have a long-distance transmission function which enables notification to the nursing staff as soon as the diaper products become soaking wet, allowing the nurses to know the current needs of their care receivers without limitations in time and space, thereby effectively enhancing the hygiene and comfort of the elderly residents.

A detailed description of the structure of the aforesaid diaper products is given below. Referring to FIG. 1, the diaper product 1 includes a first non-woven layer 11, an absorbent layer 12, a second non-woven layer 13, two sensing electrodes 14, and a plastic layer 15. One side of the first non-woven layer 11 is designed to absorb the wearer's urine. The absorbent layer 12 is composed of an absorbent material and has one side attached to the other side (hereinafter referred to as the second side) of the first non-woven layer 11 so as to absorb the urine transferred from the first non-woven layer 11 and retain the absorbed urine. One side of the second non-woven layer 13 is attached to the other side of the absorbent layer 12 and the second side of the first non-woven layer 11 such that the absorbent layer 12 is sandwiched between the first non-woven layer 11 and the second non-woven layer 13. The aforesaid side of the second non-woven layer 13 can absorb the urine leaking from the absorbent layer 12. The two sensing electrodes 14 are strip-like and each have one side attached to the other side (hereinafter referred to as the second side) of the second non-woven layer 13. The two sensing electrodes 14 are in electrical communication with each other when in contact with urine. One side of the plastic layer 15 is attached to the second side of the second non-woven layer 13 and the other side of each sensing electrode 14 such that the two sensing electrodes 14 are sandwiched between the second non-woven layer 13 and the plastic layer 15. The plastic layer 15 can hold the urine leaking from the second non-woven layer 13 and keep the urine from leaking out of the diaper product 1. Once the first non-woven layer 11, the absorbent layer 12, the second non-woven layer 13, the sensing electrodes 14, and the plastic layer 15 are sequentially sealed and secured together, the two sensing electrodes 14 are positioned within the diaper product 1 and each have one end exposed from the diaper product 1.

Referring to FIG. 1 and FIG. 2, after a user (e.g., an elderly person, a child, or a patient) puts on the diaper product 1, a nurse or caregiver (hereinafter collectively referred to as the carer) has the two alligator clips 17 of the clipping element 18 of an electrical detection device 16 clipped on the exposed ends of the two sensing electrodes 14 respectively. When the user urinates, the urine sequentially passes through the first non-woven layer 11, the absorbent layer 12, and the second non-woven layer 13 and contacts with the two sensing electrodes 14, thus short-circuiting the two sensing electrodes 14 and bringing them into electrical communication. The electrical detection device 16 now detects, through the two alligator clips 17 of the clipping element 18, the electrical communication between the two sensing electrodes 14 and sends out a warning sound to alert the carer to the fact that the urine in the diaper product 1 has reached a certain amount, thereby prompting the carer to replace the diaper product 1 with a new one.

However, the diaper product 1 described above has its drawbacks in use. With continued reference to FIG. 1 and FIG. 2, given the fact that each of the two sensing electrodes 14 has one end exposed from the diaper product 1 in order to be clipped by one of the two alligator clips 17, and that the clipping element 18 and the alligator clips 17 are structurally complicated and bulky, one who wears the diaper product 1 in bed is very likely to feel the discomfort of lying or pressing against the clipping element 18 and the alligator clips 17. Moreover, the bulky clipping element 18 and alligator clips 17, which are attached to the exposed ends of the sensing electrodes 14 only by clipping, tend to shift in position when pressed against or pulled by the user, and such a shift in position will prevent the electrical detection device 16 from accurate detection of urine and thus keep the carer from changing the diaper product 1 in a timely manner. Consequently, skin irritation or even inflammation may develop in the user' pubic area due to prolonged contact with urine. Last but not least, as the two sensing electrodes 14 are provided inside the diaper product 1, a conventional diaper manufacturer wishing to produce the diaper product 1 has to modify the existing machines and production process, which raises the production costs significantly. Installation of the two additional sensing electrodes 1 will also increase the production time.

As stated above, the existing diaper products capable of urine detection leave much room for improvement. It is, therefore, an important subject in the diaper industry to overcome the aforementioned drawbacks and design better diaper products.

### BRIEF SUMMARY OF THE INVENTION

In view of the fact that existing diaper products with a urine detecting function cannot be manufactured without costly modification of the manufacturing process of the conventional diapers and are rather uncomfortable to wearers, the inventor of the present invention conducted extensive research and numerous experiments and finally succeeded in developing a diaper structure capable of wirelessly detecting a soaking wet condition thereof and a diaper detection system of the same. The present invention solves the aforesaid problems by attaching a radio frequency identification (RFID) tag to the conventional diaper structure in order to detect a soaking wet condition thereof without having to modify the conventional diaper structure.

The first object of the present invention is to provide a diaper structure capable of wirelessly detecting a soaking wet condition thereof, thereby enabling rapid detection of a soaking wet condition of a diaper without significant modification of the conventional diaper structure. The diaper structure of the present invention includes a diaper main body, at least one communication chip, and at least one metal antenna. The diaper main body at least includes a fabric layer, an anti-leak layer, and an absorbent material layer. The fabric layer is provided on the inner side of the diaper main body and is configured for absorbing urine and allowing the urine to pass through. The anti-leak layer is provided on the outer side of the diaper main body and has an inner surface configured for blocking urine from passing through, thereby preventing the urine from leaking out of the diaper main body. The absorbent material layer is composed of an absorbent material, is provided between the fabric layer and the anti-leak layer, and is configured for absorbing the urine passing through the fabric layer and retaining the absorbed urine. When the inner surface of the anti-leak layer is in contact with the urine on the surface of the absorbent material layer, the relative permittivity of the anti-leak layer is changed. The communication chip is attached to the outer surface of the anti-leak layer and is connected to the metal antenna, which is also attached to the outer surface of the anti-leak layer. Thus, the communication chip and the metal antenna jointly form an RFID tag, and the communication chip can transmit or receive electromagnetic wave signals through the metal antenna. When the inner surface of the anti-leak layer is in contact with the urine on the surface of the absorbent material layer, the change in the relative permittivity of the anti-leak layer causes a change in the impedance value of the metal antenna and consequently a change in the wavelength of the electromagnetic wave signal transmitted by the RFID tag.

The second object of the present invention is to provide a diaper detection system for wirelessly detecting a soaking wet condition. The diaper detection system includes a diaper structure and a signal reader, wherein the diaper structure is the one described in the first object. The signal reader is configured for emitting a detecting electromagnetic wave to the RFID tag and stores a setting value, which is the wavelength of the electromagnetic wave that the metal antenna should transmit when the inner surface of the anti-leak layer is not wetted. As stated previously, when the user urinates in the diaper main body such that the absorbent material layer is soaking wet and further wets the inner surface of the anti-leak layer, the impedance value of the metal antenna is changed. Once the RFID tag receives the detecting electromagnetic wave, and the communication chip generates a response signal accordingly, the response signal will transmit a response electromagnetic wave because of the change in the impedance value of the metal antenna. When the wavelength of the response electromagnetic wave is different from the setting value, the signal reader determines that the diaper main body is soaking wet. The signal reader may further send out a warning (e.g., a sound or a flashing light) in order for the carer to change the diaper immediately.

The third object of the present invention is to provide another diaper detection system for wirelessly detecting a soaking wet condition. This diaper detection system also includes a diaper structure and a signal reader, and the diaper structure is still the one described in the first object. The signal reader is configured for emitting a detecting electromagnetic wave to the RFID tag and stores a setting value, which is the wavelength of the electromagnetic wave that the metal antenna should transmit when the inner surface of the anti-leak layer is wetted. As stated previously, when the user urinates in the diaper main body such that the absorbent material layer is soaking wet and further wets the inner surface of the anti-leak layer, the impedance value of the metal antenna is changed. Once the RFID tag receives the detecting electromagnetic wave, and the communication chip generates a response signal accordingly, the response signal will transmit a response electromagnetic wave because of the change in the impedance value of the metal antenna. When the wavelength of the response electromagnetic wave is equal to the setting value, the signal reader determines that the diaper main body is soaking wet. The signal reader may further send out a warning (e.g., a sound or a flashing light) in order for the carer to change the diaper immediately.

### BRIEF DESCRIPTION OF THE SEVERAL VIEWS OF THE DRAWINGS

The objects, as well as the technical features and their effects, of the present invention will be best understood by referring to the following detailed description of some illustrative embodiments in conjunction with the accompanying drawings, in which:

FIG. 1 is an exploded perspective view of a conventional diaper product;

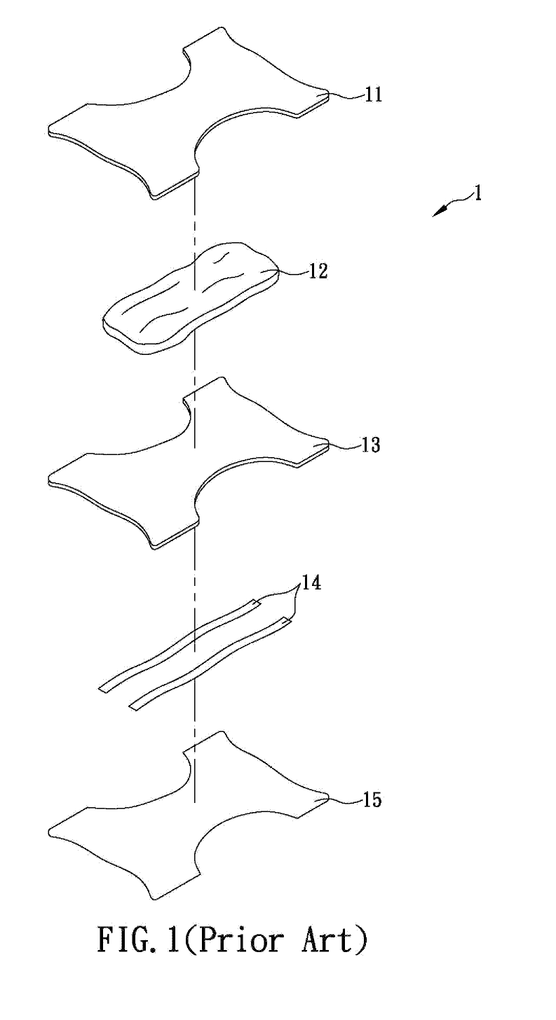

FIG. 2 schematically shows the diaper product depicted in FIG. 1 and an electrical detection device;

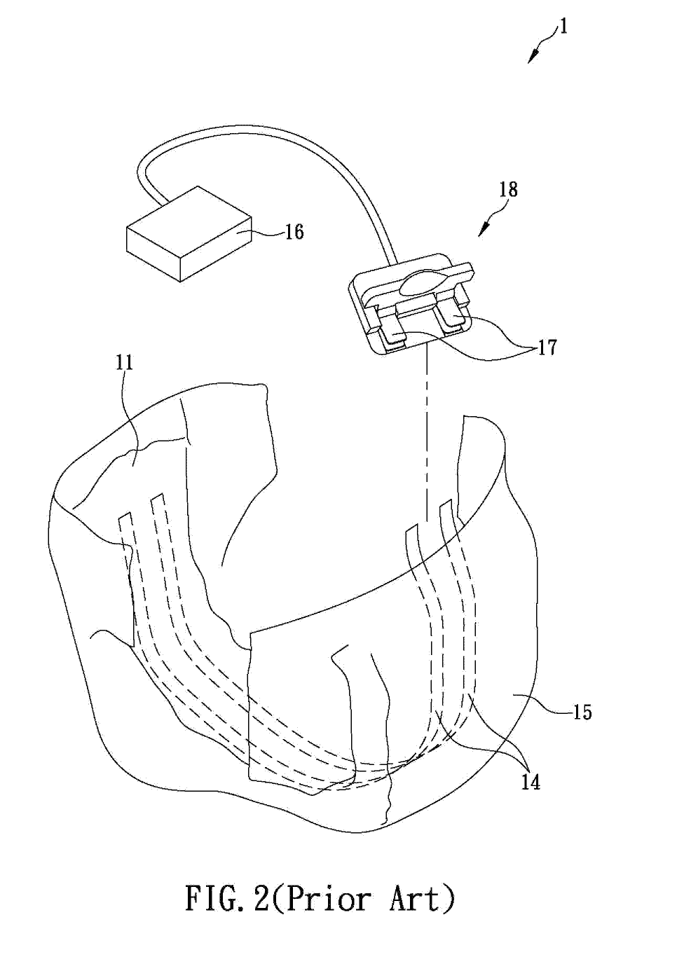

FIG. 3 schematically shows the diaper structure of the present invention;

FIG. 4 schematically shows the diaper detection system of the present invention; and

FIG. 5 is a plot showing simulated impedance matching of the diaper detection system of the present invention.

### DETAILED DESCRIPTION OF THE INVENTION

Radio frequency identification (RFID) is a technique in which data are transmitted via electromagnetic waves and which is suitable for identification purposes. Generally speaking, RFID is a non-contact automatic identification method involving a reader and a tag and works in the following manner. A communication chip (e.g., an integrated circuit) having a storage memory is disposed on a tag, and the data in the communication chip can be transmitted to a reader through an antenna in the tag for the purpose of identification. It is of particular interest here that the properties of the antenna will change according to the relative permittivity of the substrate material to which the antenna is attached, as expressed by the equations:

$$v = f λ ; v = \frac{c}{\sqrt{{ɛ}_{r}}} ;$$

where ν is the velocity of an electromagnetic wave signal travelling through the material, c is the speed of light, εᵣ is the relative permittivity of the material, f is the frequency of the electromagnetic wave signal, and λ is the wavelength of the electromagnetic wave signal. According to the equations, the greater the relative permittivity of the material is, the shorter the wavelength will be. In other words, when the relative permittivity of the substrate material to which the antenna is attached is changed, the wavelength of the electromagnetic wave signal is, too. The inventor of the present invention incorporated the foregoing properties into the design of the disclosed diaper structure capable of wirelessly detecting a soaking wet condition thereof and a diaper detection system of the same.

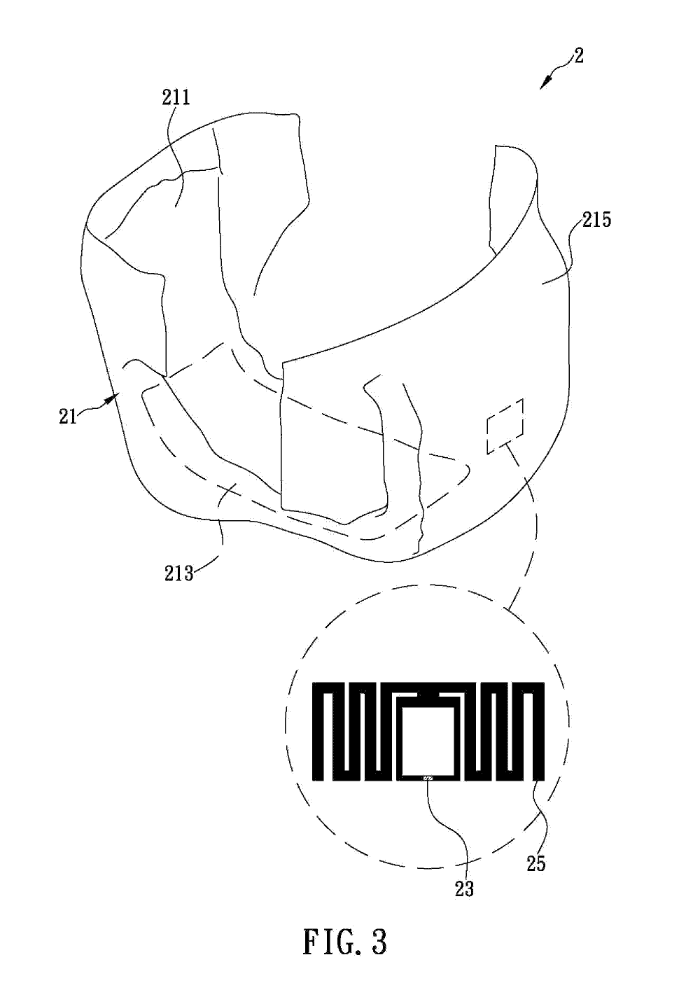

In an embodiment of the present invention as shown in FIG. 3, the diaper structure 2 includes a diaper main body 21, at least one communication chip 23, and at least one metal antenna 25. The diaper main body 21 at least includes a fabric layer 211, an absorbent material layer 213, and an anti-leak layer 215. The fabric layer 211 is provided on the inner side of the diaper main body 21 and is configured to absorb urine while allowing the urine to pass through. The anti-leak layer 215 is provided on the outer side of the diaper main body 21 and has an inner surface configured for blocking urine from passing through, thereby preventing the urine from leaking out of the diaper main body 21. The absorbent material layer 213 is composed of an absorbent material, is provided between the fabric layer 211 and the anti-leak layer 215, and is configured to absorb the urine passing through the fabric layer 211 and retain the urine absorbed. Should the inner surface of the anti-leak layer 215 be in contact with the urine on the surface of the absorbent material layer 213, the relative permittivity of the anti-leak layer 215 will be changed.

It should be pointed out that the diaper main body 21 in the embodiment shown in FIG. 3 is merely the essential structure of a diaper. The present invention is applicable to all kinds of diapers on the market, provided that the diaper main body has the fabric layer 211, the absorbent material layer 213, and the anti-leak layer 215, and that the fabric layer 211 and the anti-leak layer 215 are provided on the inner and outer sides of the diaper main body respectively, regardless of whether any additional material (e.g., multiple layers of non-woven fabric) is provided between the fabric layer 211 and the absorbent material layer 213 or between the absorbent material layer 213 and the anti-leak layer 215.

Referring to FIG. 3, the communication chip 23 is attached to the outer surface of the anti-leak layer 215, and so is the metal antenna 25. The metal antenna 25 is connected to the communication chip 23 such that the metal antenna 25 and the communication chip 23 jointly form an RFID tag. It should be noted that the shape of the metal antenna 25 illustrated in FIG. 3 is provided by way of example only. In other embodiments of the present invention, the shape of the metal antenna 25 may be adjusted according to product requirements. The communication chip 23 can transmit or receive electromagnetic wave signals through the metal antenna 25. As stated above, when the relative permittivity of the substrate material to which the metal antenna 25 is attached is changed, the wavelength of the electromagnetic wave signal transmitted through the metal antenna 25 is also changed. Since the metal antenna 25 is attached to the anti-leak layer 215, and the relative permittivity of the anti-leak layer 215 will be changed when the inner surface of the anti-leak layer 215 is in contact with the urine on the surface of the absorbent material layer 213, the wavelength of the electromagnetic wave signal transmitted by the RFID tag will vary with the relative permittivity of the anti-leak layer 215. In other words, the wavelength of the electromagnetic wave signal transmitted by the RFID tag when the inner surface of the anti-leak layer 215 is not wetted with urine will be totally different from when the inner surface of the anti-leak layer 215 is wetted with urine.

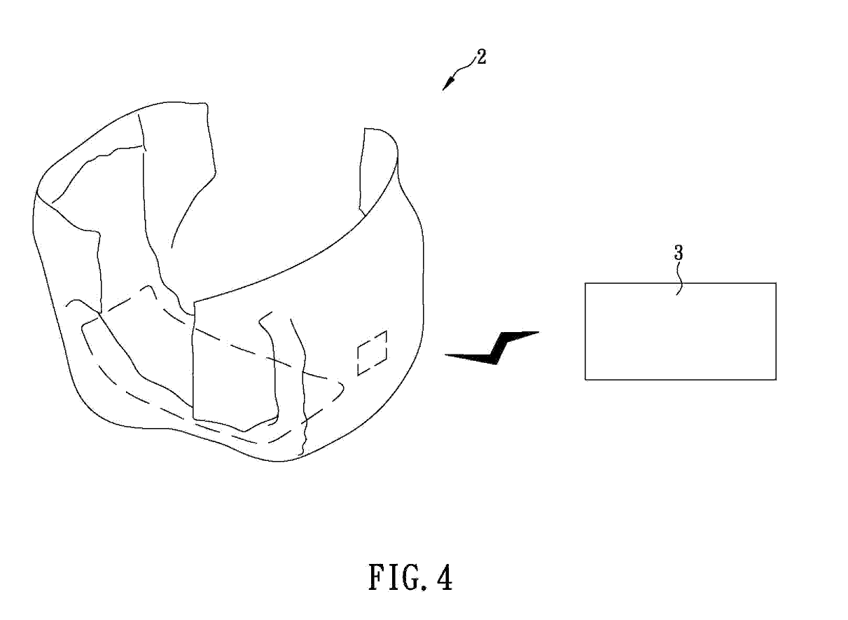

Referring to FIG. 3 and FIG. 4, a signal reader 3 is provided for emitting a detecting electromagnetic wave to the RFID tag. The signal reader 3 stores a setting value therein, and the setting value is the numerical value of the wavelength of the electromagnetic wave to be transmitted by the metal antenna 25 when the inner surface of the anti-leak layer 21 is not wetted. In this embodiment, if the user urinates in the diaper main body 21 such that the absorbent material layer 213 is soaking wet and in turn wets the inner surface of the anti-leak layer 215, the impedance value of the metal antenna 25 attached to the outer surface of the anti-leak layer 215 will be changed, and the response signal generated by the communication chip 23 after the RFID tag receives the detecting electromagnetic wave will transmit a response electromagnetic wave due to the change in the impedance value of the metal antenna 25, wherein the wavelength of the response electromagnetic wave is different from the setting value (i.e., the wavelength of the electromagnetic wave that the metal antenna 25 should transmit when the inner surface of the anti-leak layer 215 is not wetted). After receiving the response electromagnetic wave, the signal reader 3 can readily compare the wavelength of the received response electromagnetic wave with the setting value stored in the signal reader 3 and rapidly determine whether the diaper main body 21 is soaking wet. The nursing staff of a medical or nursing institution only have to check the data of the signal reader 3 or be prompted by a warning (e.g., a sound or a flashing light) sent out by the signal reader 3, and they will know when and where a diaper needs to be changed, without having to open up and then fasten each care receiver's diaper only to check whether it is wet. In other embodiments of the present invention, the content of the setting value may be differently set to make the present invention even more convenient. For example, the setting value may be the numerical value of the wavelength of the electromagnetic wave to be transmitted by the metal antenna when the inner surface of the anti-leak layer 215 is wetted.

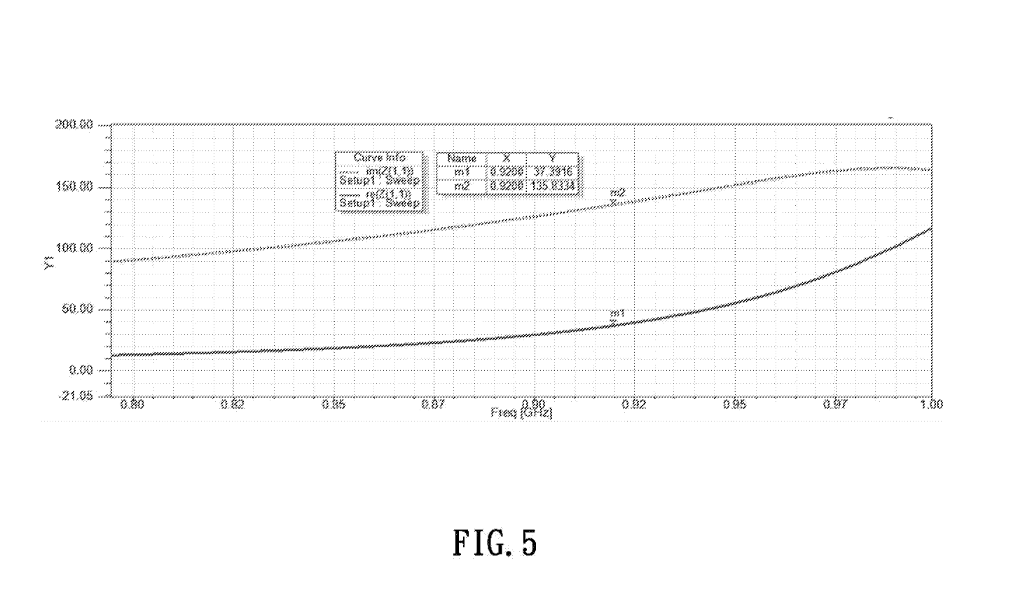

According to the above, referring back to FIG. 3 and FIG. 4, when the diaper main body 21 absorbs and becomes saturated with urine, the relative permittivity of the diaper main body 21 rises significantly, which alters the impedance, and consequently the impedance matching, of the metal antenna 25 of the RFID tag tightly attached to the diaper main body 21. Moreover, in the foregoing embodiment, the metal antenna 25 is directly matched to the communication chip 23, without using a matching network. Therefore, production costs can be reduced, and the area which will otherwise be taken up by the electronic components of a matching network is saved. In addition, the impedance seen from the feed point of the communication chip 23 toward the metal antenna 25 has a relatively low real-part resistance (lower than 50 ohms) and a relatively high imaginary-part inductive reactance (higher than 100), as shown in the plot of simulated impedance matching of FIG. 5, in which the real-part resistance is in solid line and the imaginary-part inductive reactance is in dotted line. It can be clearly seen in FIG. 5 that the real-part resistance is well below 50 ohms whereas the imaginary-part inductive reactance is higher than 100, meaning the metal antenna 25 can be directly matched to a commercially available communication chip 23. The same simulation test also shows that, when the diaper main body 21 attached with the RFID tag of the present invention is wrapped around the user's bottom, the RFID tag can successfully receive the energy transmitted from the RFID signal reader 3 and send information back to the signal reader 3 to prompt the carer to change diapers.

While the invention herein disclosed has been described by means of specific embodiments, numerous modifications and variations could be made thereto by those skilled in the art without departing from the scope of the invention set forth in the claims.

What is claimed is:

1. A diaper structure capable of wirelessly detecting a soaking wet condition thereof, comprising:
a diaper main body comprising a fabric layer, an anti-leak layer, and an absorbent material layer, the fabric layer being provided on an inner side of the diaper main body, the anti-leak layer being provided on an outer side of the diaper main body and having an inner surface configured for blocking urine from leaking through, the absorbent material layer being composed of an absorbent material, provided between the fabric layer and the anti-leak layer, and configured for absorbing urine passing through the fabric layer, the anti-leak layer having a relative permittivity which is changed when the inner surface of the anti-leak layer is in contact with urine on a surface of the absorbent material layer;
at least one communication chip attached to an outer surface of the anti-leak layer; and
at least one metal antenna attached to the outer surface of the anti-leak layer, the metal antenna being connected to the communication chip such that the metal antenna and the communication chip form a radio frequency identification (RFID) tag, wherein the communication chip can transmit or receive electromagnetic wave signals through the metal antenna, and when the inner surface of the anti-leak layer is in contact with the urine on the surface of the absorbent material layer, a change in the relative permittivity of the anti-leak layer causes a change in an impedance value of the metal antenna and consequently a change in the wavelength of an electromagnetic wave signal transmitted by the RFID tag.

2. A diaper detection system for wirelessly detecting a soaking wet condition, comprising:
a diaper main body comprising a fabric layer, an anti-leak layer, and an absorbent material layer, the fabric layer being provided on an inner side of the diaper main body, the anti-leak layer being provided on an outer side of the diaper main body and having an inner surface configured for blocking urine from leaking through, the absorbent material layer being composed of an absorbent material, provided between the fabric layer and the anti-leak layer, and configured for absorbing urine passing through the fabric layer, the anti-leak layer having a relative permittivity which is changed when the inner surface of the anti-leak layer is in contact with urine on a surface of the absorbent material layer;
at least one communication chip attached to an outer surface of the anti-leak layer;
at least one metal antenna attached to the outer surface of the anti-leak layer, the metal antenna being connected to the communication chip such that the metal antenna and the communication chip form a radio frequency identification (RFID) tag, wherein the communication chip can transmit or receive electromagnetic wave signals through the metal antenna, and when the inner surface of the anti-leak layer is in contact with the urine on the surface of the absorbent material layer, a change in the relative permittivity of the anti-leak layer causes a change in an impedance value of the metal antenna and consequently a change in the wavelength of an electromagnetic wave signal transmitted by the RFID tag; and
a signal reader for emitting a detecting electromagnetic wave to the RFID tag, wherein when a user urinates in the diaper main body such that the absorbent material layer becomes soaking wet and wets the inner surface of the anti-leak layer, causing the change in the impedance value of the metal antenna, a response signal generated by the communication chip after the RFID tag receives the detecting electromagnetic wave transmits a response electromagnetic wave due to the change in the impedance value of the metal antenna, the response electromagnetic wave having a wavelength different from a setting value, the setting value being the wavelength of an electromagnetic wave the metal antenna should transmit when the inner surface of the anti-leak layer is not wetted.

3. The diaper detection system of claim 2, wherein the signal reader sends out a warning upon determining that the wavelength of the response electromagnetic wave is different from the setting value.

4. A diaper detection system for wirelessly detecting a soaking wet condition, comprising:
a diaper main body comprising a fabric layer, an anti-leak layer, and an absorbent material layer, the fabric layer being provided on an inner side of the diaper main body, the anti-leak layer being provided on an outer side of the diaper main body and having an inner surface configured for blocking urine from leaking through, the absorbent material layer being composed of an absorbent material, provided between the fabric layer and the anti-leak layer, and configured for absorbing urine passing through the fabric layer, the anti-leak layer having a relative permittivity which is changed when the inner surface of the anti-leak layer is in contact with urine on a surface of the absorbent material layer;
at least one communication chip attached to an outer surface of the anti-leak layer;
at least one metal antenna attached to the outer surface of the anti-leak layer, the metal antenna being connected to the communication chip such that the metal antenna and the communication chip form a radio frequency identification (RFID) tag, wherein the communication chip can transmit or receive electromagnetic wave signals through the metal antenna, and when the inner surface of the anti-leak layer is in contact with the urine on the surface of the absorbent material layer, a change in the relative permittivity of the anti-leak layer causes a change in an impedance value of the metal antenna and consequently a change in the wavelength of an electromagnetic wave signal transmitted by the RFID tag; and
a signal reader for emitting a detecting electromagnetic wave to the RFID tag, wherein when a user urinates in the diaper main body such that the absorbent material layer becomes soaking wet and wets the inner surface of the anti-leak layer, causing the change in the impedance value of the metal antenna, a response signal generated by the communication chip after the RFID tag receives the detecting electromagnetic wave transmits a response electromagnetic wave due to the change in the impedance value of the metal antenna, the response electromagnetic wave having a wavelength equal to a setting value, the setting value being the wavelength of an electromagnetic wave the metal antenna should transmit when the inner surface of the anti-leak layer is wetted.

5. The diaper detection system of claim 4, wherein the signal reader sends out a warning upon determining that the wavelength of the response electromagnetic wave is equal to the setting value.
